(19) United States
(12) Patent Application Publication
(10) Pub. No.: US 2019/0046351 A1
(43) Pub. Date: Feb. 14, 2019

(54) PROTECTIVE BAND HAVING CHIPS

(71) Applicant: GHI FU INT'L TECHNOLOGY CO., LTD., Chang Hua County (TW)

(72) Inventor: Li-Chi LIN, CHANG HUA COUNTY (TW)

(21) Appl. No.: 15/675,413

(22) Filed: Aug. 11, 2017

(51) Int. Cl. A61F 7/10; A61F 7/08; A61F 7/02

(52) U.S. Cl. CPC A61F 7/103; A61F 7/086; A61F 2007/023; A61F 2007/0252; A61F 7/02

(57) ABSTRACT

A protective band having chips includes a band body having a plurality of compartments. The compartments are provided with chip sets, respectively. Each chip set has two fixing films and a plurality of chips wrapped between the fixing films. The fixing films each have a fixed end fixed to a corresponding one of the compartments and a free end accommodated in the corresponding compartment. The protective band can be attached to the user's skin to radiate far infrared rays. Through the fixed ends of the fixing films fixed to the corresponding compartment, the fixing films are maintained at a predetermined position to enhance the wearing comfort and maintain the radiation effect of the chip sets.

### FIELD OF THE INVENTION

The present invention relates to a protective band, and more particularly to a protective band having chips.

### BACKGROUND OF THE INVENTION

These days, the living standard is improved and people pay more attention to health. Many far infrared products are developed for nursing. When far infrared ray is close to the human body, the far infrared ray will radiate heat energy to be absorbed by the water molecules of the human body. The large water molecules are decomposed into small water molecules, resulting in deviational vibration to promote blood circulation and to enhance metabolism and to provide the effects of sterilization and deodorization. In order to make far infrared close to the human body, a far infrared protective band is developed accordingly to be attached to the skin of the human body. A conventional protective band includes a main body having a plurality of compartments thereon. Chip sets are provided in the compartments, respectively. Each chip of the chip sets is coated with an energy coating layer. The energy coating layer is activated to radiate far infrared rays having a preferred wavelength to promote blood circulation.

However, the chip sets are placed in the compartments. When the user moves or changes the posture, the chip sets will move, bend, or fold, causing the displacement of the chip sets. The user may feel uncomfortable, and it is necessary to adjust the position of the chip set manually. When the chip set is displaced, in addition to reducing the coverage of the far infrared rays to affect the effect of the product, the chips may incline to get contact with the skin of the human body, causing the user's discomfort. Accordingly, the inventor of the present invention has devoted himself based on his many years of practical experiences to solve these problems.

### SUMMARY OF THE INVENTION

The primary object of the present invention is to provide a protective band having chips. The protective band includes chip sets positioned in the protective band.

In order to achieve the aforesaid object, the protective band of the present invention comprises a band body and a plurality of chip sets. The band body has a first connecting portion at one end thereof and a second connecting portion at another end thereof. The band body has a plurality of compartments. Each chip set has two fixing films and a plurality of chips wrapped between the fixing films. The chip sets are disposed in the compartments, respectively. The fixing films of the chip sets each having a fixed end fixed to a corresponding one of the compartments and a free end accommodated in the corresponding compartment.

Through the fixed ends of the fixing films fixed to the corresponding compartment, the fixing films are maintained at a predetermined position to enhance the wearing comfort and maintain the radiation effect of the chip sets.

### BRIEF DESCRIPTION OF THE DRAWINGS

FIG. 1 is a perspective view in accordance with a first embodiment of the present invention;

FIG. 2 is a partial exploded view in accordance with the first embodiment of the present invention;

FIG. 3 is a sectional view in accordance with the first embodiment of the present invention;

FIG. 4 is a schematic view in accordance with the first embodiment of the present invention when in use, showing that the present invention is applied to a neck guard;

FIG. 5 is a schematic view in accordance with a second embodiment of the present invention when in use, showing that the present invention is applied to a wrist guard; and

FIG. 6 is a schematic view in accordance with the first embodiment of the present invention when in use, showing that the present invention is applied to a waist guard.

### DETAILED DESCRIPTION OF THE PREFERRED EMBODIMENTS

Embodiments of the present invention will now be described, by way of example only, with reference to the accompanying drawings.

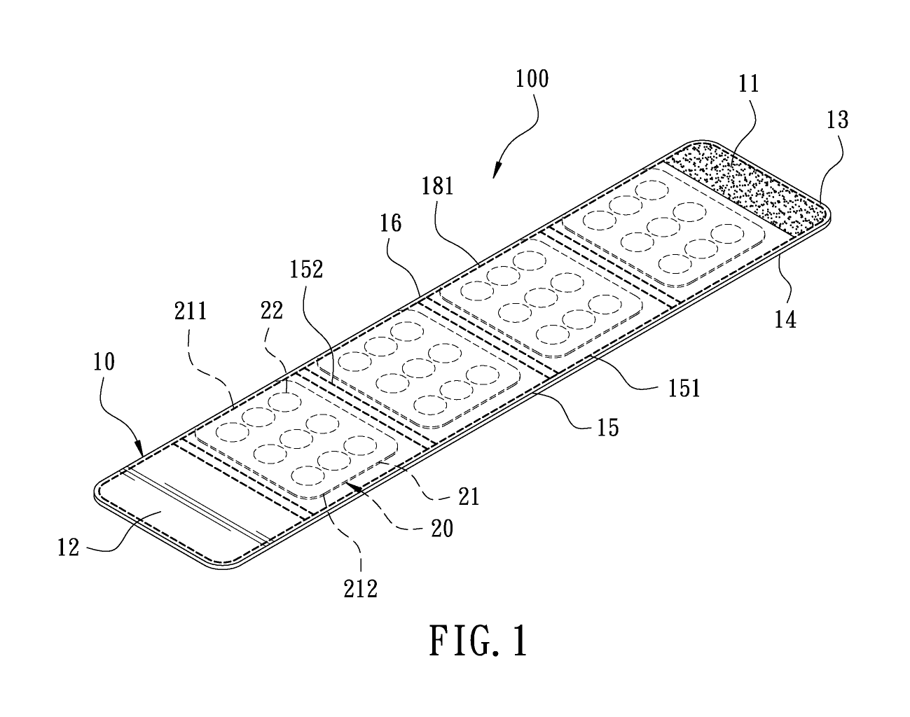

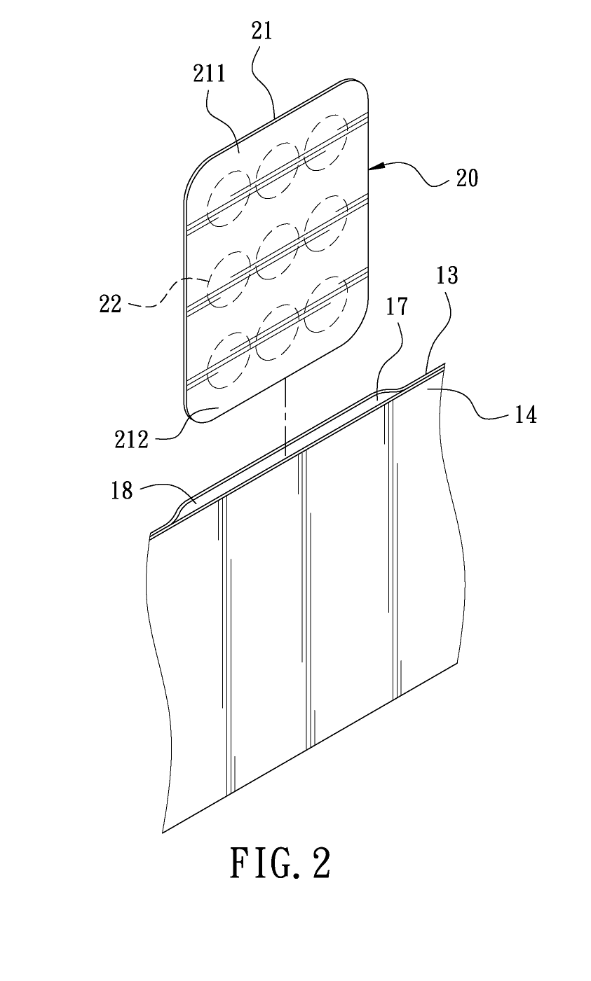

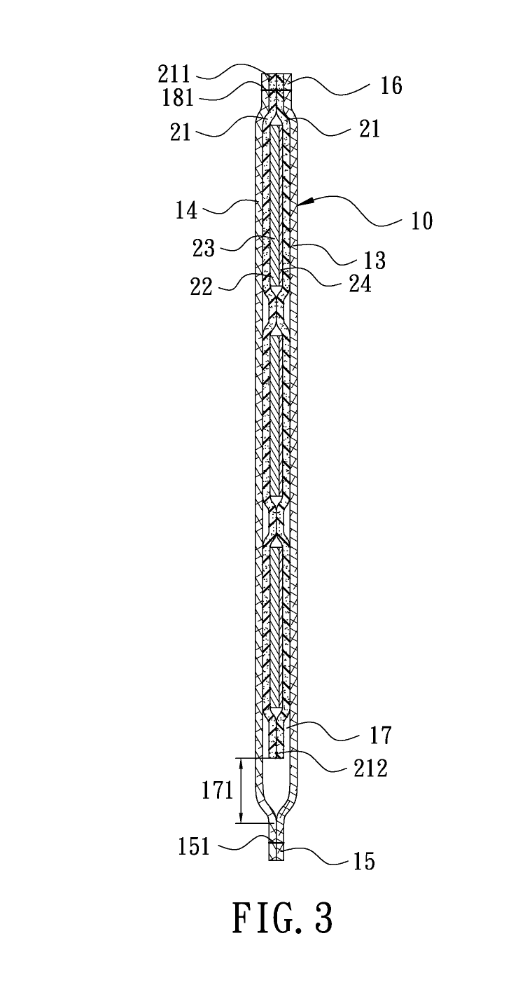

FIG. 1 is a perspective view in accordance with a first embodiment of the present invention. FIG. 2 is a partial exploded view in accordance with the first embodiment of the present invention. FIG. 3 is a sectional view in accordance with the first embodiment of the present invention. The present invention discloses a protective band 100 having chips. The protective band 100 comprises a band body 10 and a plurality of chip sets 20.

The band body 10 has a first connecting portion 11 at one end thereof and a second connecting portion 12 at another end thereof. The first connecting portion 11 is a male fastening tape, and the second connecting portion 12 is a female fastening tape. The band body 10 has a first fabric body 13 and a second fabric body 14. The band body 10 has a first side 15 and a second side 16. The band body 10 is sewn to form a first stitch line 151 at the first side 15 to fix the first fabric body 13 and the second fabric body 14 together and a plurality of second stitch lines 152 perpendicular to the first side 15 and spaced from each other to fix the first fabric body 13 and the second fabric body 14 together so that a plurality of compartments 17 are formed between the first fabric body 13 and the second fabric body 14, and the second side 16 of the band body 10 is formed with mouths 18 communicating with the compartments 17 respectively.

Each chip set 20 has two fixing films 21 and a plurality of chips 22 wrapped between the fixing films 21. The fixing films 21 are made of flexible EVA, PE, TPR or TPU, etc. The fixing films 21 each have a fixed end 211 and a free end 212. The fixed ends 211 of the fixing films 21 are located at a corresponding one of the mouths 18 of the band body 10 and sewn to form a third stitch line 181 so as to fix the fixed ends 211 of the fixing films 21 to the corresponding mouth 18 of the band body 10 and close the corresponding mouth 18 of the band body 10. The free ends 212 of the fixing films 21 are accommodated in a corresponding one of the compartments 17 and disposed corresponding to the first side 15 of the band body 10. An interval 171 is defined between the free ends 212 of the fixing films 21 and the first side 15 of the band body 10. In this embodiment, the second side 16 of the band body 10 is disposed above the first side 15. When the fixed ends 211 of the fixing films 21 are fixed to the second side 16, the free ends 212 of the fixing films 21 are naturally sagged by gravity. In addition, each of the chips 22 includes a metal substrate 23. The metal substrate 23 is coated with a functional layer 24. The functional layer 24 is made of a material selected from one of copper, carbon, silicon, manganese, phosphorus, sulfur, nickel, germanium, chromium, molybdenum, titanium, vanadium, calcium and silicon or a mixture thereof.

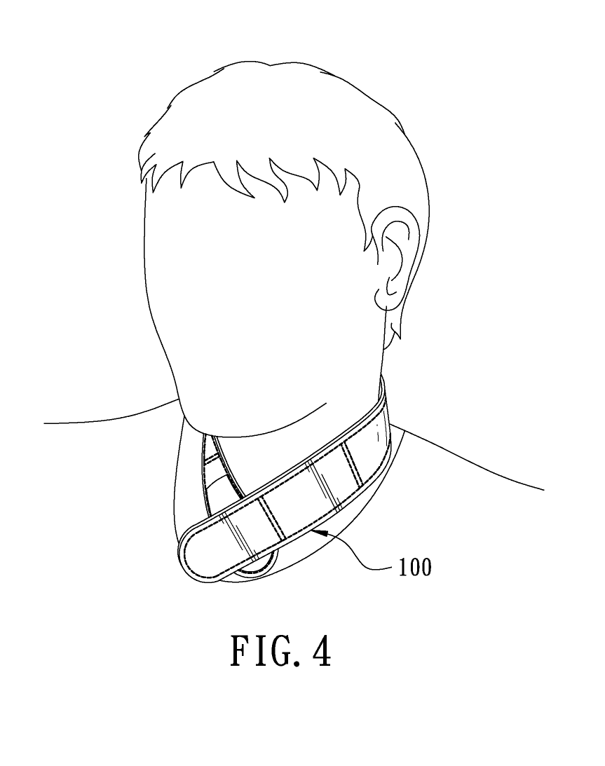

FIG. 4 is a schematic view in accordance with the first embodiment of the present invention when in use, showing that the present invention is applied to a neck guard. The protective band 100 having chips is placed on the user's neck. Since the fixing films 21 are made of a flexible material, it is always in contact with the user's skin along with the movement of the neck. The temperature of the human body is about 32-37° C., which can activate the functional layers 24 of the chips 22 to radiate optimum far infrared waves of 8-14 μm to promote blood circulation of the neck.

The fixed ends 211 of the fixing films 21 are sewn and fixed to the corresponding mouth 18 of the band body 10, so that the fixed ends 211 of the fixing films 21 enable the free ends 212 of the fixing films 21 to move freely when the user is in various postures (such as, stoop down), and the fixing films 21 are inclined to get contact with the skin of the human body. When the user returns to the standing posture, the free ends of the fixing films 21 are free to be sagged by gravity and attached to the skin of the human body. Thus, the fixing films 21 are maintained at a predetermined position to enhance the wearing comfort and maintain the radiation effect of the chip sets 20.

The interval 171 is formed between the free ends 212 of the fixing films 21 and the first side 15 of the band body 10. The free ends 212 of the fixing films 21 won't get contact with the first side 15 of the band body 10 and won't be compressed and deformed when the band body 10 is subjected to stress and deformed, maintaining a comfortable effect for the user. When the user returns to the standing posture, the free ends of the fixing films 21 are free to be sagged by gravity and attached to the skin of the human body. Thus, the fixing films 21 are maintained at a predetermined position to enhance the wearing comfort and maintain the radiation effect of the chip sets 20.

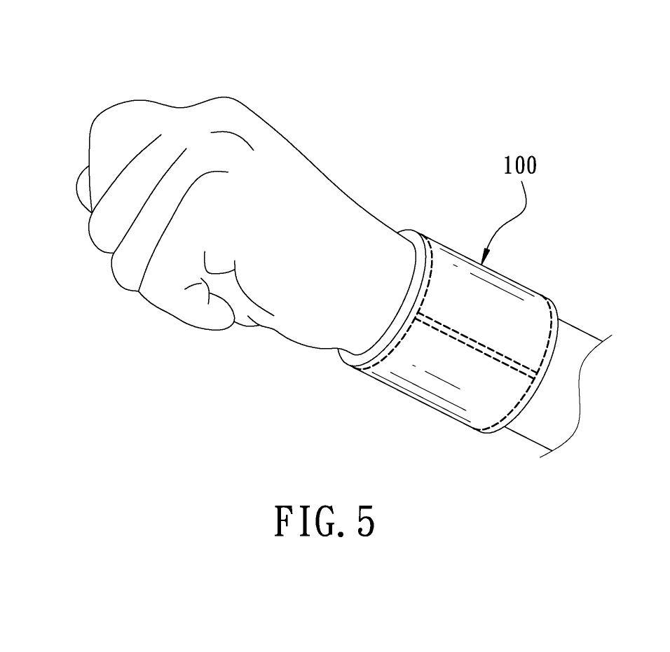

FIG. 5 is a schematic view in accordance with a second embodiment of the present invention when in use, showing that the present invention is applied to a wrist guard. The second embodiment of the present invention differs from the foregoing first embodiment in that the protective band 100 having chips is placed on the wrist to promote blood circulation of the wrist.

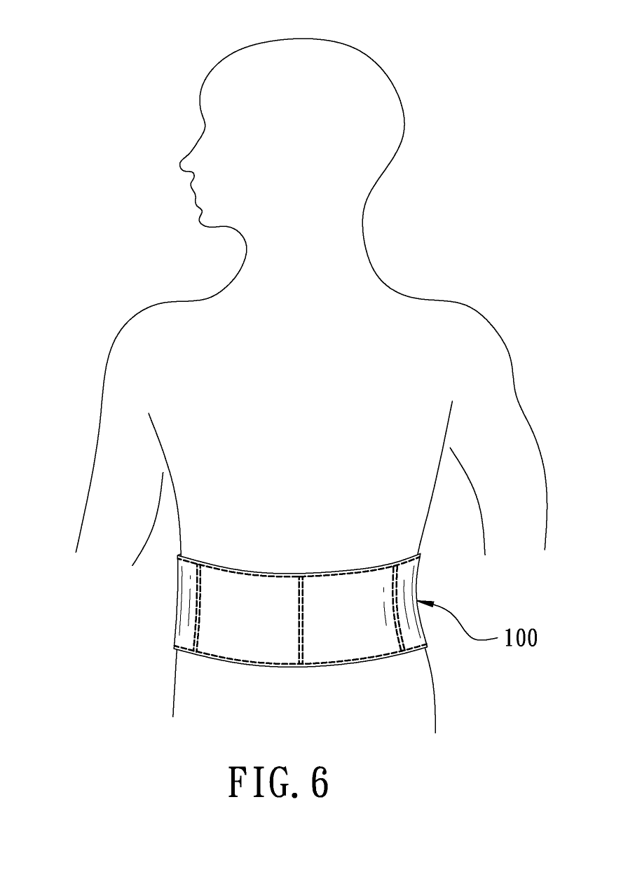

FIG. 6 is a schematic view in accordance with a third embodiment of the present invention when in use, showing that the present invention is applied to a waist guard. The third embodiment of the present invention differs from the foregoing first embodiment in that the protective band 100 having chips is placed around the waist to promote blood circulation of the waist.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A protective band, comprising:
a band body, having a first connecting portion at one end thereof and a second connecting portion at another end thereof, the band body having a plurality of compartments; and
a plurality of chip sets, each chip set having two fixing films and a plurality of chips wrapped between the fixing films, the chip sets being disposed in the compartments respectively, the fixing films of the chip sets each having a fixed end fixed to a corresponding one of the compartments and a free end accommodated in the corresponding compartment.

2. The protective band as claimed in claim 1, wherein each of the chips includes a metal substrate, and the metal substrate is coated with a functional layer.

3. The protective band as claimed in claim 2, wherein the functional layer is made of a material selected from one of includes copper, carbon, silicon, manganese, phosphorus, sulfur, nickel, germanium, chromium, molybdenum, titanium, vanadium, calcium and silicon or a mixture thereof.

4. The protective band as claimed in claim 1, wherein the band body has a first fabric body and a second fabric body, the band body has a first side and a second side, the band body is sewn to form a first stitch line at the first side to fix the first fabric body and the second fabric body together and a plurality of second stitch lines perpendicular to the first side and spaced from each other to fix the first fabric body and the second fabric body together so that the plurality of compartments are formed between the first fabric body and the second fabric body, and the second side of the band body is formed with mouths communicating with the compartments respectively.

5. The protective band as claimed in claim 4, wherein the fixed ends of the fixing films are located at a corresponding one of the mouths of the band body and sewn to form a third stitch line so as to fix the fixed ends of the fixing films to the corresponding mouth of the band body and close the corresponding mouth of the band body, and the free ends of the fixing films are disposed corresponding to the first side of the band body.

6. The protective band as claimed in claim 5, wherein an interval is defined between the free ends of the fixing films and the first side of the band body.

7. The protective band as claimed in claim 4, wherein the second side of the band body is disposed above the first side, when the fixed ends of the fixing films are fixed to the second side, the free ends of the fixing films are naturally sagged by gravity.
